(19) United States
(12) Patent Application Publication
(10) Pub. No.: US 2011/0165277 A1
(43) Pub. Date: Jul. 7, 2011

(54) Biological Materials and Uses Thereof

(76) Inventors: Ahmed Ali, Swansea (GB); Charles James Newbold, Aberystwyth (GB); Kenton Hart, Aberystwyth (GB)

(21) Appl. No.: 12/999,910

(22) PCT Filed: Jun. 19, 2009
(86) PCT No.: PCT/GB09/01547

§ 371 Date: Mar. 4, 2011

(30) Foreign Application Priority Data
Jun. 20, 2008 (GB) 0811362.3

(51) Int. Cl. A61K 36/18; A61K 31/045; A61P 1/00

(52) U.S. Cl. 424/769; 514/729

(57) ABSTRACT

There is provided the use of a sandalwood extract or a sandalwood analogue as an additive to animal foodstuff for the reduction of methane production, reduction of bacterial mediated protein breakdown and reduces bacterial growth in the stomach. There are also provided food products and methods of making food products incorporating sandalwood extracts or sandalwood analogues.

The present invention relates to additives for animal foodstuffs and to methods for beneficially regulating ruminant digestion.

The supply of antibiotic growth promoters to farm animals is a well known method in agriculture for increasing the yield of meat or diary produce. The term “antibiotic growth promoter” is used to describe any medicine that destroys or inhibits bacteria and is administered at a low, sub-therapeutic dose. Infectious agents reduce the yield of farmed food animals and, to control these, the administration of sub-therapeutic antibiotics and antimicrobial agents has been shown to be effective. Although the mechanism underpinning their action is unclear, it is believed that the antibiotics suppress sensitive populations of bacteria in the intestines.

It has been estimated that as much as six percent of the net energy in the pig diet could be lost due to microbial fermentation in the intestine. If the microbial population could be better controlled, it is possible that the lost energy could be diverted to growth. Similarly upwards of ten percent of the energy in the diet of cattle and sheep is lost through the production of methane during microbial fermentation, decreasing methane production in the rumen using antimicrobial agents not only diverts this energy to meat and milk production but also lower the production of this harmful greenhouse gas. Whatever the mechanism of action, the use of growth promoters results in an improvement in daily growth rates between one and ten percent resulting in meat of a better quality, with less fat and increased protein content.

Currently, there is some unease surrounding the use of growth promoters in animals destined for meat production, as overuse of any antibiotic over a period of time may lead to the local bacterial populations becoming resistant to the antibiotic. Human health is potentially also directly affected through residues of an antibiotic in meat, which may cause side-effects.

In response to growing concerns regarding the effects of antibiotic growth promoters on human health, in January 2006 the European Union effectively prohibited on the use of antibiotics as growth promoters in animal agriculture. As such, there is currently an unsatisfied demand for alternatives to antibiotics. Livestock producers must find alternative means of obtaining similar production benefits to maintain and improve the standards and quantities of livestock products but also to maintain the profitability and competitiveness of the livestock industry. Some countries around the world, including the USA, do not currently have restrictions on the use of antibiotics as growth promoters in animal agriculture, however such restrictions may exist in the near future and there is also a need to improve those livestock that are treated with antibiotics. Ways must also be found to improve the healthiness and safety of animal products reaching the consumer, including those from organic farming.

There are also important social issues concerning the removal of antibiotic growth promoters including possible higher cost of production being passed to the consumer and the risks to both human and animal health through the greater prevalence of pathogenic organisms in the animal. These factors will drive the rapid acceptance of new products, providing they are efficacious.

We have identified a plant extract (selected from a screening of almost 2500 such compounds) that beneficially manipulates digestion in the gut of ruminant livestock to promote the economic, safe and environmentally friendly production of meat and milk. Specifically the extract of interest prevents the growth of E. coli 0157 and Listeria monocytogenes in the rumen; reduces the rate of protein breakdown (allowing more protein to be absorbed by the gut of the animal and thus boosting production); and decreases the emission of,the important greenhouse gas methane.

A first aspect of the invention provides the use of a sandalwood extract or a sandalwood analogue as an additive to animal foodstuff.

Sandalwood extract is an essential oil extracted from trees in the genus Santalum. The extract has commonly been used for incense, aromatherapy and as an ingredient in perfume. Sandalwood essential oil has also been used in medicine, mostly as a urogenital and skin antiseptic. Its main component, santalols, has known antimicrobial properties. However, it has not previously been suggested that sandalwood extract could beneficially manipulate ruminant digestion in the gut of ruminant livestock.

As disclosed herein, the inventors have determined that sandalwood extract prevents the growth of E. coli 0157 and Listeria monocytogenes in the digestive system of ruminants. In addition there is also a reduction in protein breakdown in the rumen, which allows more protein to be absorbed thus boosting meat and milk production, and a decrease in the emission of methane from the rumen. Therefore sandalwood extract can be used as an additive to animal foodstuff in order to bring about important and beneficial changes in ruminant digestion.

It is important to note that the beneficial properties of sandalwood extract are not common to all extracts or compounds having antimicrobial properties. For example, during our trials we have tested some 2500 plant extracts including numerous essential oil compounds without finding a comparable extract.

By “sandalwood extract” we include where the extract is the essential oil prepared from trees of the genus Santalum. The extract can be obtained commercially from very many sources. Examples of sandalwood extract that can be used in the present invention include: Sandalwood oil manufactured by SAFC (e.g. W30,050-0 lot no. 03722CC-396) and Sandalwood oil manufactured by Fluka (355263/1 lot no. 52706264), Sandalwood oil from Swiss Herbal Remedies (B/N 540).

By “sandalwood analogue” we mean a compound or mixture of compounds that resembles sandalwood on the basis of smell (see, for example, Bieri et al (2004) Chem Senses. 29(6):483-7 Olfactory receptor neuron profiling using sandalwood odorants). Such analogues include natural analogues extracted from natural sources such as essential oils and synthetic sandalwood replacement compounds or mixtures. Examples of synthetic replacements include Javanol™ (e.g. from Givaudan lot nos. 9000591570 and 90000635339) and Santaliff™ (e.g. from International Flavour and Fragrances lot no. R000485362). Further alternatives are readily available and a number of these alternatives are discussed further in the examples.

Such compositions contain chemical compounds having the structures:

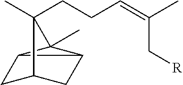

where:
α-Santalol: R═OH

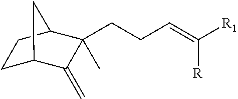

where
for β-Santalol : R═CH₂OH and R1═H
for E-cis-epi-β-Santalol: R═H and R1═CH₂OH
   and for those chemical analogues based on campholenic aldehyde:

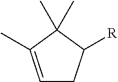

where:
R=3 methyl pentanol=Sandalore:
R=3-methyl pent-4-en-2-ol=Ebanol
R=(E)-2-methylbut-2-en-1-ol=Santaliff
R=(E)-2-ethylbut-2-en-1-ol=Bacdanol and Sanjinol isomers

Other R groups are possible, as will be apparent to those skilled in the art.

As discussed above, sandalwood extract or analogues thereof can be used to bring about these important and beneficial changes in ruminant digestion. Hence it is preferred that it used as an additive in ruminant diets although it may also be beneficial in monogastric animals such as horses.

A ruminant is an animal that digests its food in two steps: first by eating the raw material and regurgitating a semi-digested form known as a cud, then eating the cud, a process called ruminating. Ruminants have a stomach with four chambers,.which are the rumen, reticulum, omasum and abomasum. In the first two chambers, the rumen and the reticulum, the food is mixed with saliva and separates into layers of solid and liquid material. Solids clump together to form the cud (or bolus). The cud is then regurgitated, chewed slowly to completely mix it with saliva, which further breaks down fibers. Fibre, especially cellulose, is broken down into glucose in these chambers by symbiotic bacteria, protozoa and fungi. The broken-down fiber, which is now in the liquid part of the contents, then passes through the rumen into the next stomach chamber, the omasum, where water is removed. After this the digesting food is moved to the last chamber, the abomasum. The food in the abomasum is digested much like it would be in the human stomach. It is finally sent to the small intestine, where the absorption of the nutrients occurs.

Almost all the glucose produced by the breaking down of cellulose is used by the symbiotic bacteria. Ruminants get their energy from the volatile fatty acids produced by these bacteria: lactic acid, propionic acid and butyric acid.

Ruminant animals include include cattle, goats, sheep, camels, llamas, giraffes, bison, buffalo, deer, wildebeest and antelope. Preferably the sandalwood extract is used as an additive for foodstuffs for domesticated livestock such as cattle, goats, sheep or llamas.

The sandalwood extract or analogue can be added to the foodstuff after the foodstuff has been prepared or during preparation of the foodstuff.

Preferably the foodstuff is suitable for administration to an animal, particularly a ruminant or horse. Whilst note exclusive examples of foodstuffs to which the sandalwood extract can be added include: total mixed rations (TMR) ensiled and fresh forage, grains, manufactured concentrates protein supplement and by-products. However it is likely that the preferred method of addition would be via premixes and mineral and vitamin supplements either incorporated into diets of off or on farm.

The amount of sandalwood extract or analogue used in the invention is between 0.025 g per day and 50 g per day. Preferred amounts are between 0.5 and 50 g/day for larger ruminants e.g. cattle, preferably 5 g/day. Further preferred amounts are between 0.025 g and 2.5 g/day for small ruminants such as sheep preferably 0.25 g/day.

These amounts can alternatively be expressed as 25 mg/kg-50 g/kg, preferably 500 mg/kg.

A further method of the invention provides a method for reducing the growth of pathogenic bacteria in the digestive system of a ruminant or horse comprising supplying the ruminant or horse with sandalwood extract or an analogue thereof.

As set out in the accompanying examples, sandalwood extract or an analogue thereof acts within the digestive system to reduce pathogenic bacterial growth. The reduction in pathogenic bacterial growth caused by the method of the invention is beneficial as there is also a reduction in bacterial levels in meat derived from ruminants. Since some bacteria pose significant hazards to human health, for example E. coli, then the method of the invention can be useful in improving the hygiene of meat. A preferred embodiment of this aspect of the invention is wherein bacterial growth is reduced in the rumen.

By “reducing” we include that the sandalwood extract reduces bacterial growth by 25% in comparison to a reference sample.

Preferably the method of this aspect of the invention reduces E. coli and/or Listeria monocytogenes growth.

Sandalwood extract or an analogue thereof is supplied to a ruminant or horse as part of the method of this aspect of the invention. Examples of sandalwood extract as an additive are described above and are suitable for use in the method of the invention. Preferably the method of this aspect of the invention uses the animal foodstuff set out above.

A further aspect of the invention provides a method of increasing meat and/or milk production from a ruminant or horse comprising supplying the ruminant or horse with sandalwood extract or an analogue thereof.

A still further method of the invention provides a method for reducing protein breakdown in the digestive system of a ruminant or horse comprising supplying the ruminant or horse with sandalwood extract or an analogue thereof.

By “increasing meat and/or milk production” we include that the sandalwood extract or an analogue thereof increases meat and/or milk production by at least 5-10% of the weight or volume of the product, in comparison to a reference sample.

By “reducing protein breakdown in the digestive system” we include that the sandalwood extract or an analogue thereof reduces protein breakdown in the digestive system by 10-20% in comparison to a reference sample.

As set out in the accompanying examples, sandalwood extract or an analogue thereof acts within the digestive system to decrease in protein degradation in the gut and hence increase protein absorption by the animal. The increase in protein absorption leads to increased meat and/or milk production from the ruminant or horse and/or reduced feeding costs.

An embodiment of the method of the invention is wherein protein breakdown is reduced in the rumen.

A further method of the invention provides a method of reducing methane emission by a ruminant or horse comprising supplying the ruminant or horse with sandalwood extract or an analogue thereof.

Sandalwood extract or an analogue thereof can be supplied to a ruminant or horse as part of the methods of these aspects of the invention. Examples of sandalwood extract or an analogue thereof as an additive are described above and are suitable for use in these methods of the invention. Preferably these methods of the invention use the animal foodstuff set out above.

A further aspect of the invention provides the use of sandalwood extract or an analogue thereof to reduce the growth of pathogenic bacteria in the digestive system of a ruminant or horse.

A further aspect of the invention provides the use of sandalwood extract or an analogue thereof to increase meat and/or milk production from a ruminant or horse.

A further aspect of the invention provides the use of sandalwood extract or an analogue thereof to reduce protein breakdown in the digestive system of a ruminant or horse.

A further aspect of the invention provides the use of sandalwood extract or an analogue thereof to reduce methane emission by a ruminant or horse.

Preferably the sandalwood analogue of any aspect of the invention has the structure:

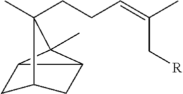

where:
R═OH

Alternatively, the sandalwood analogue of any aspect of the invention has the structure:

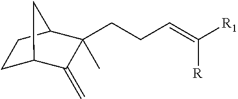

where
R═CH₂OH and R1═H; or
R═H and R1═CH₂OH

Further alternatively, the sandalwood analogue of any aspect of the invention has the structure:

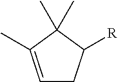

where:

R=3 methyl pentanol, 3-methyl pent-4-en-2-ol, (E)-2-methylbut-2-en-1-ol, or (E)-2-ethylbut-2-en-1-ol

The invention will now be described in more detail, for the purposes of illustration only, in the following Examples and Figures.

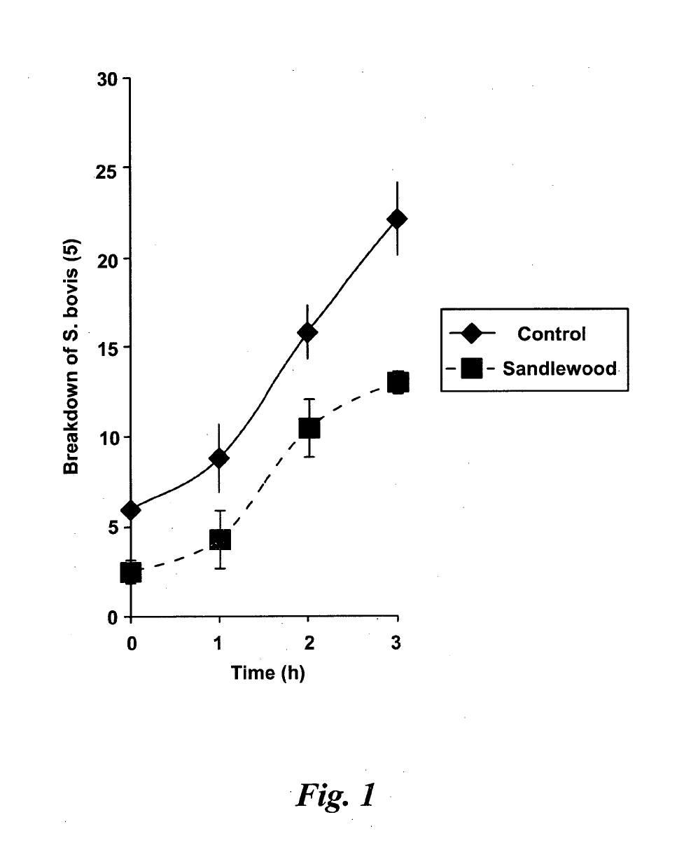

FIG. 1—The effect of 500 μg/ml Sandalwood Oil on the breakdown of S. bovis protein in rumen fluid.

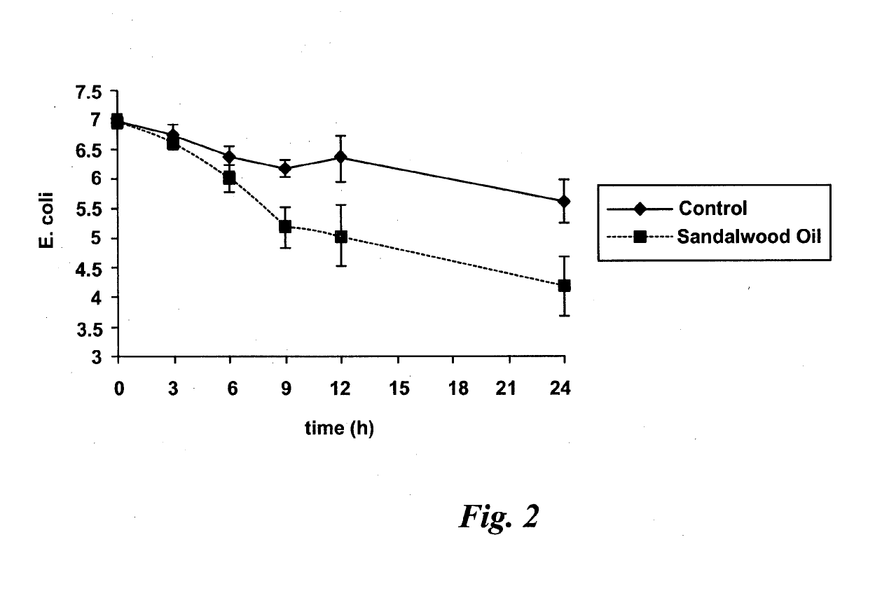

FIG. 2—Effect of Sandalwood Oil on the decline of E. coli 0157 in the rumen simulation fermentor Rusitec

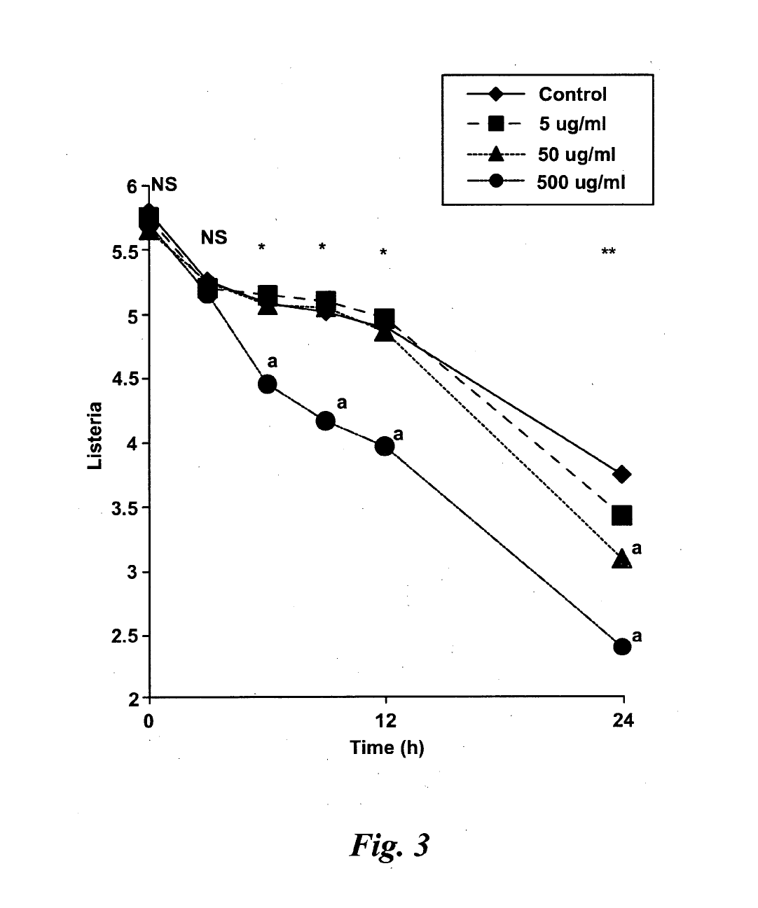

FIG. 3—Effect of Sandalwood Oil on the decline of Listeria monocytogenes in the rumen simulation fermentor Rusitec

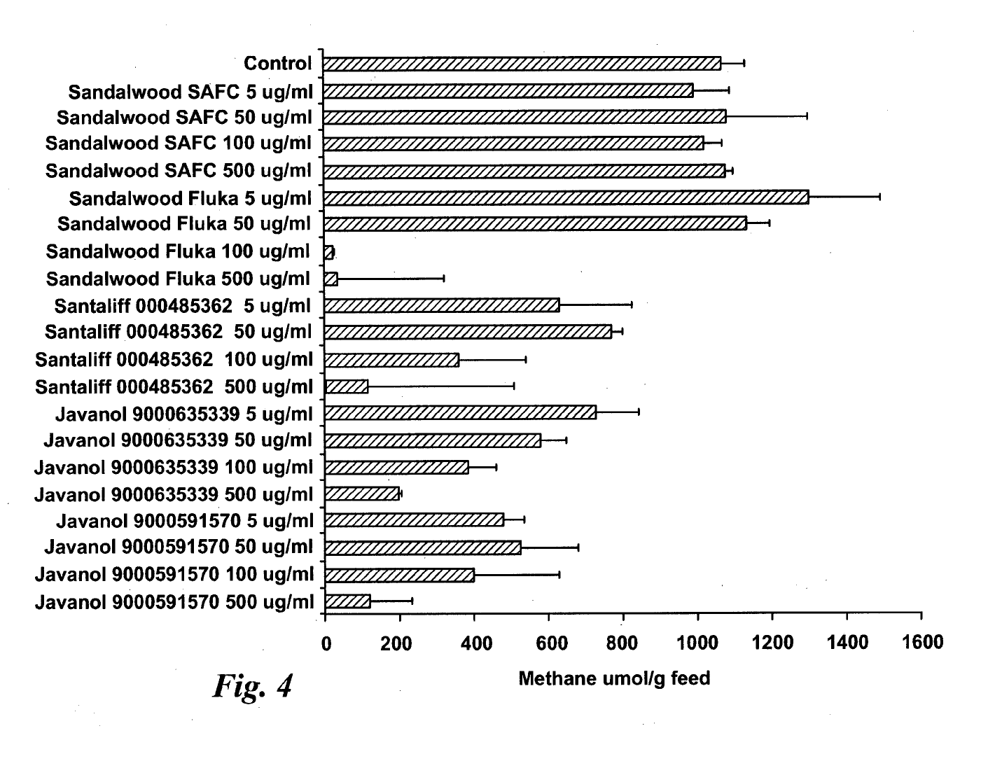

FIG. 4—shows the results of the methane production, and demonstrates that the Sandalwood oil from Fluka and both batches of Javanol and Santalifff when compared to the control experiments significantly decreased methane

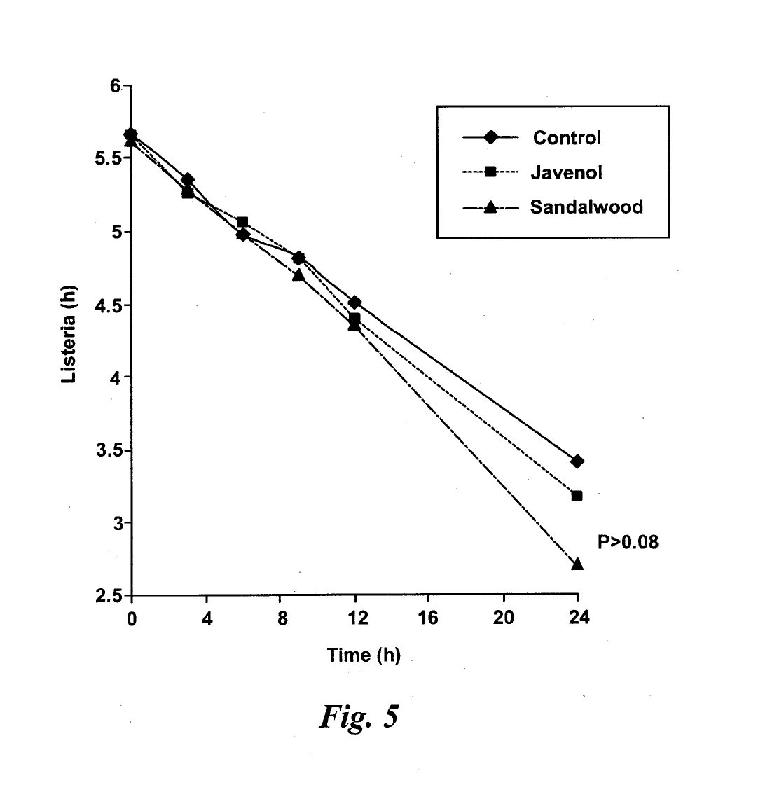

FIG. 5—Sandalwood oil and Javanol effect on methane production.

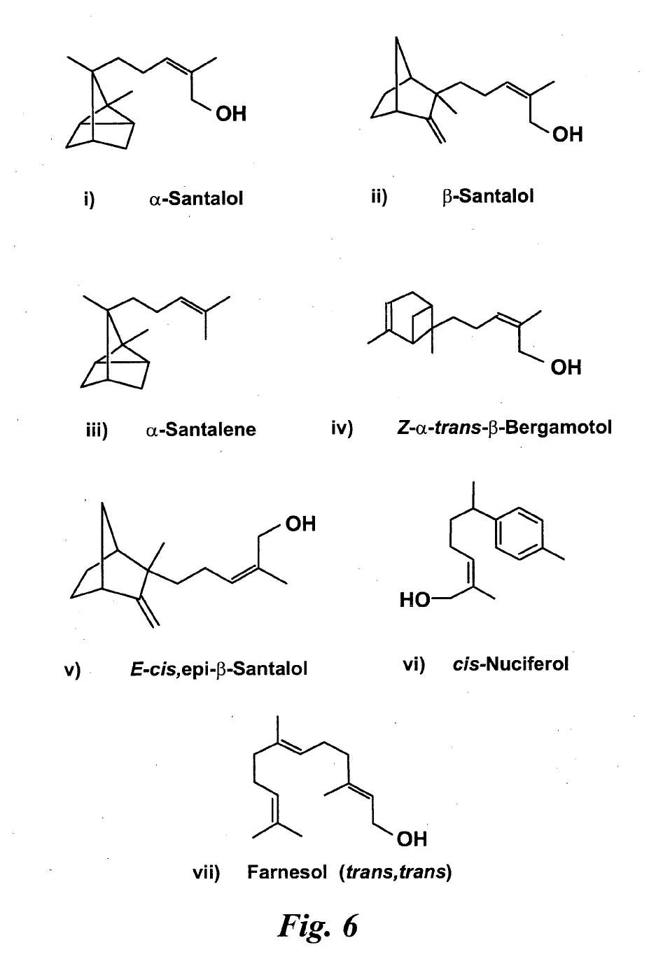

FIG. 6—Chemical structures of:

i) α-Santalol

(5-(2,3-dimethyl-tricyclo[2.2.1.02,6]hept-3-yl)-2-methyl-pent-2-en-1-ol)

ii) β-Santalol

(2-methyl-5-(2-methyl-3-methylene- bicyclo[2.2.1]hept-2-yl-pent-2-en-1-ol)

iii) α-Santalene

(1,7-dimethyl-7-(4-methyl-3-pentenyl)-tricyclo[2.2.1.0(2,6)]heptane)

iv) Z-α-trans-β-Bergamotol

(1S-(1a.,5a.,6a.(Z)-5-(2,6-dimethylbicyclo(3.1.1)hept-2-en-6-yl)-2-methyl-2-pentem-1-ol

v) E-cis,epi-β-Santalol

(2-methyl-5-((1R,2R,4S)-2-methyl-3 -methylenebicyclo(2.2.1)hept-2-yl)-(2Z)-2-penten-1-ol)

vi) cis-Nuciferol

(S—(Z)-2-methyl-6-(4-methylphenyl)-2-hepten-1-ol)

vii) Farnesol (trans,trans)

(E,E) 3,7,11-trimethyl-2,6,10-dodectrien-1-ol

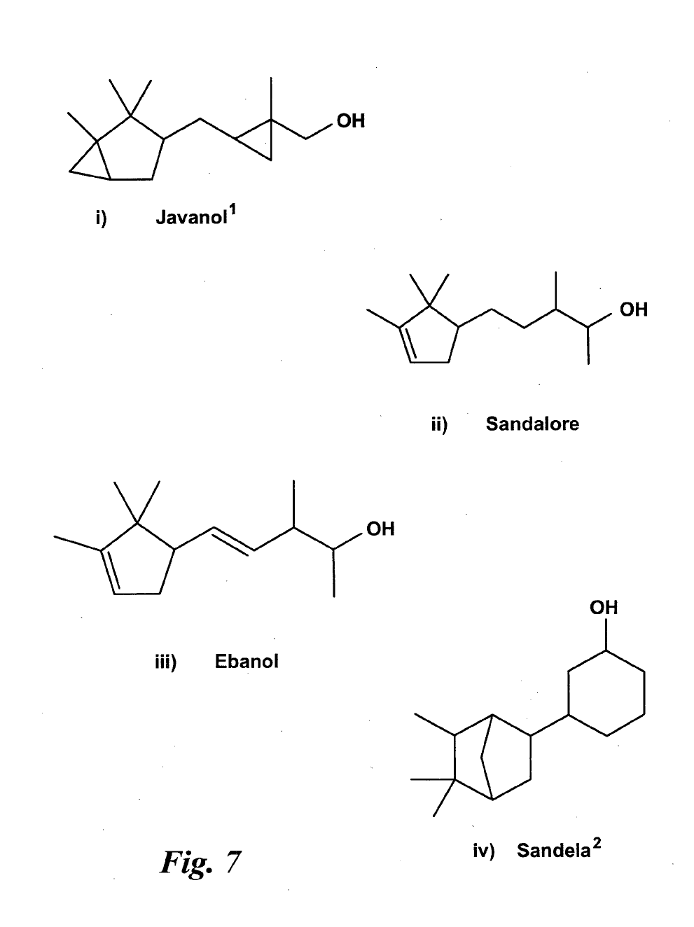

FIG. 7—Chemical structures of:

i) Javanol¹

(1-Methyl-2-(1,2,2-trimethylbicyclo[3.1.0]hex-3-ylmethyl)cyclopropyl)methanol

ii) Sandalore

5-(2,2,3-Trimethyl-3-cyclopentenhyl)-3-methylpentan-2-ol

iii) Ebanol

3-Methyl-5-(2,2,3-trimethyl-3-cyclopenten-1-yl)-4-penten-2-ol

iv) Sandela²

4-(5,5,6-Trimethylbicyclo[2.2.1]hept-2-yl)cyclohexan-1-ol

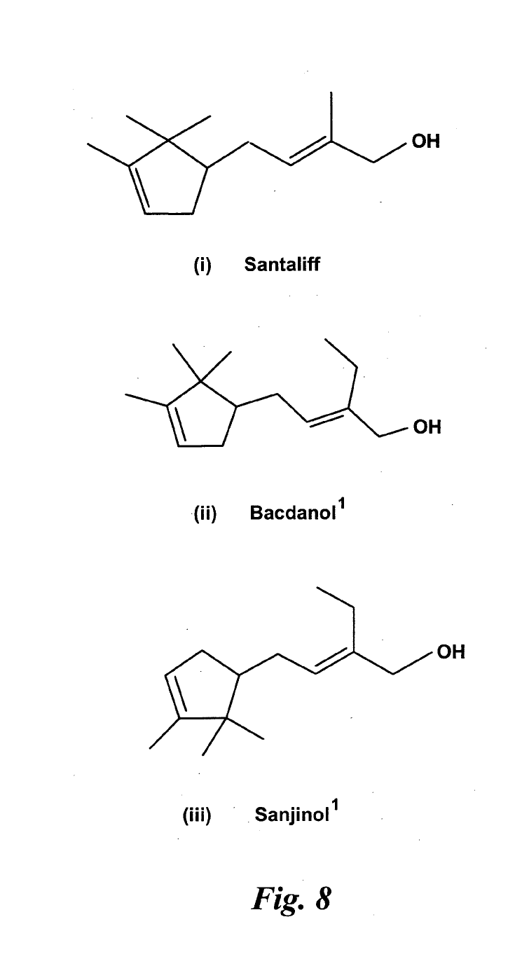

FIG. 8—Chemical structures of:

(i) Santaliff

(2-Methyl-4-(2,2,3-trimethyl-3-cyclopenten-1-yl)-2-buten-1-ol)

(ii) Bacdanol¹

(2-ethyl-4-(2,2,3-trimethyl-3-cyclopenten-1-yl)-2-buten-1-ol)

(iii) Sanjinol¹

(2-ethyl-4(2,2,3-trimethyl-3-cyclopentenyl)-2-buten-1-ol)

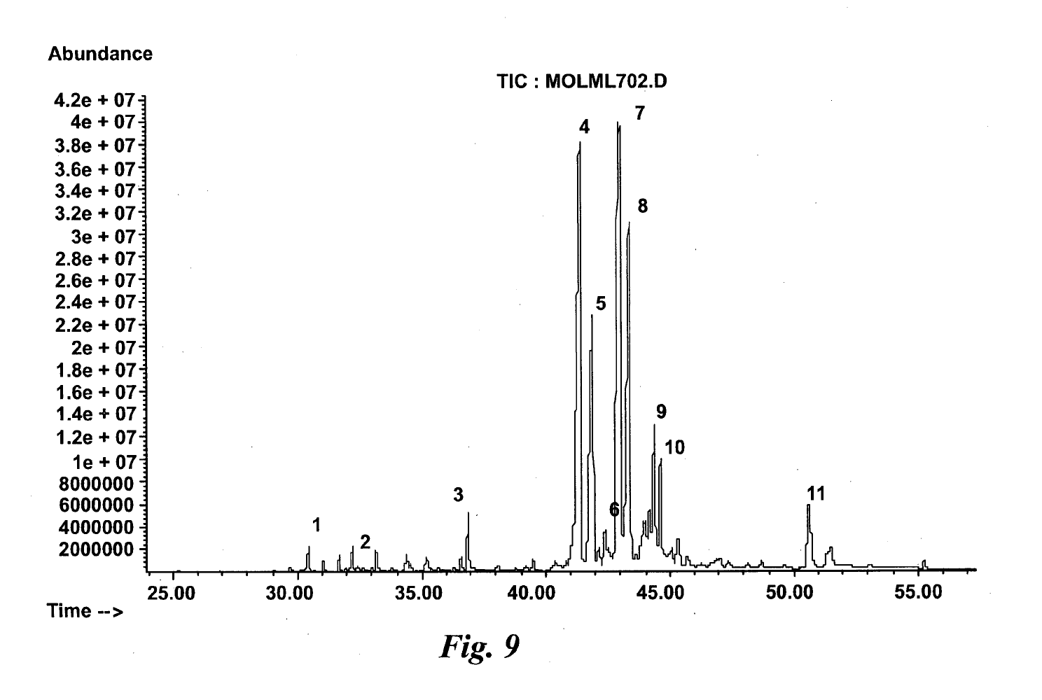

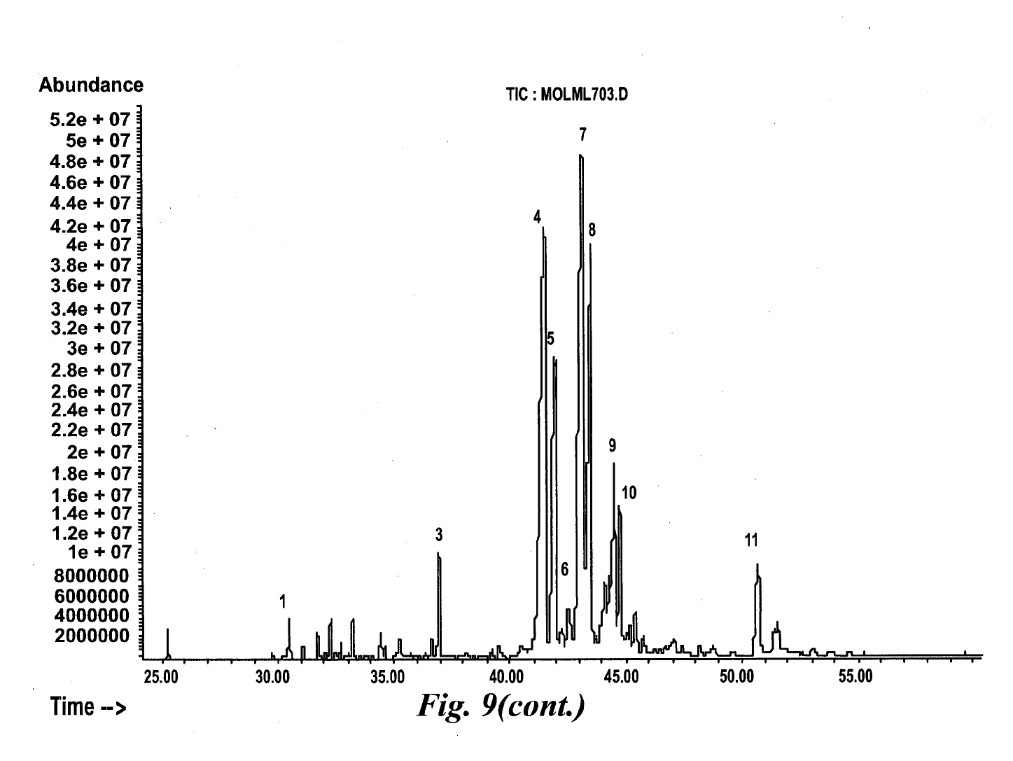

FIG. 9 Sandalwood oil (Swiss Herbal Remedies)

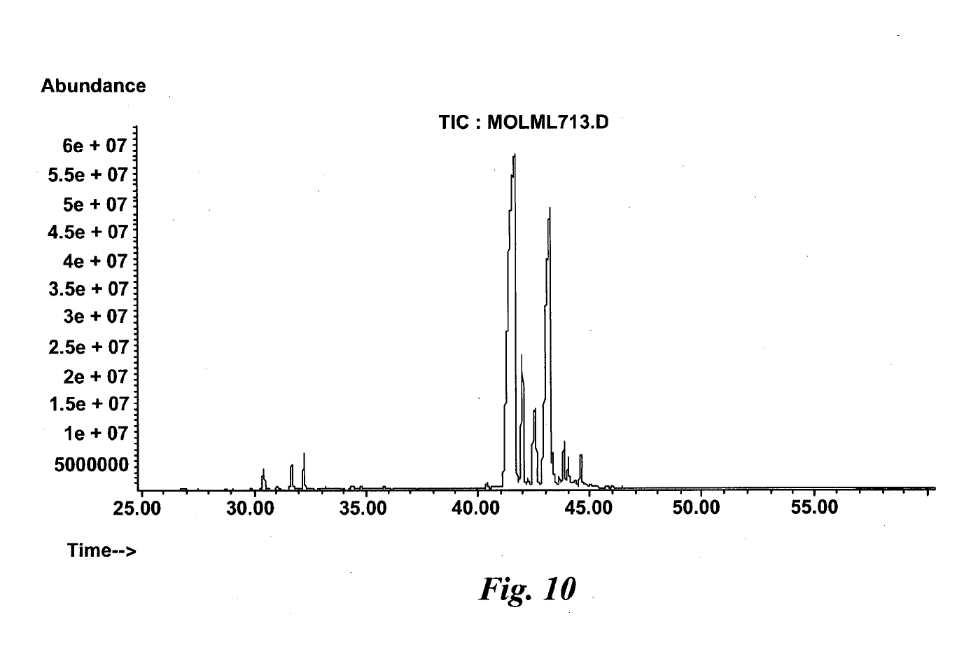

FIG. 10—Sandalwood Oil (sample D, SAFC)

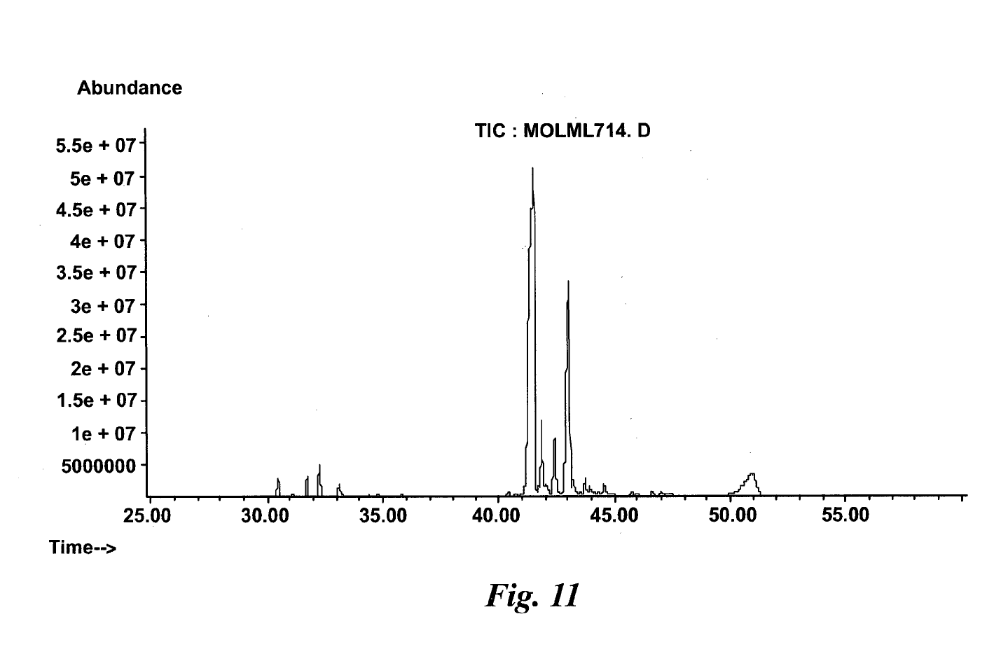

FIG. 11—Sandalwood Oil Sample (sample E, Fluka)

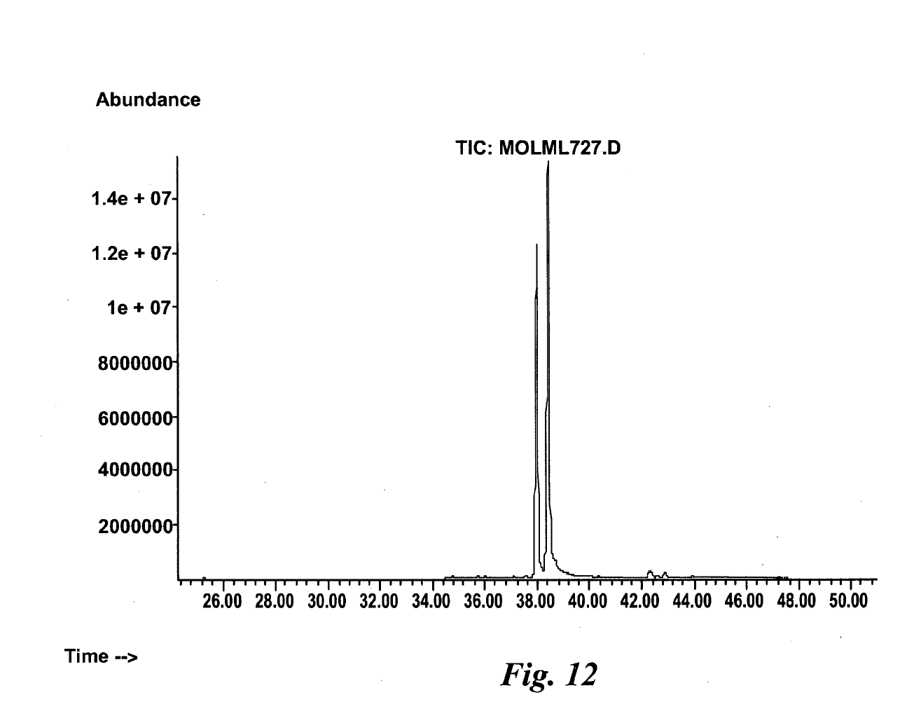

FIG. 12—Purity of chemical analogues by gc-ms: Javanol (sample A)

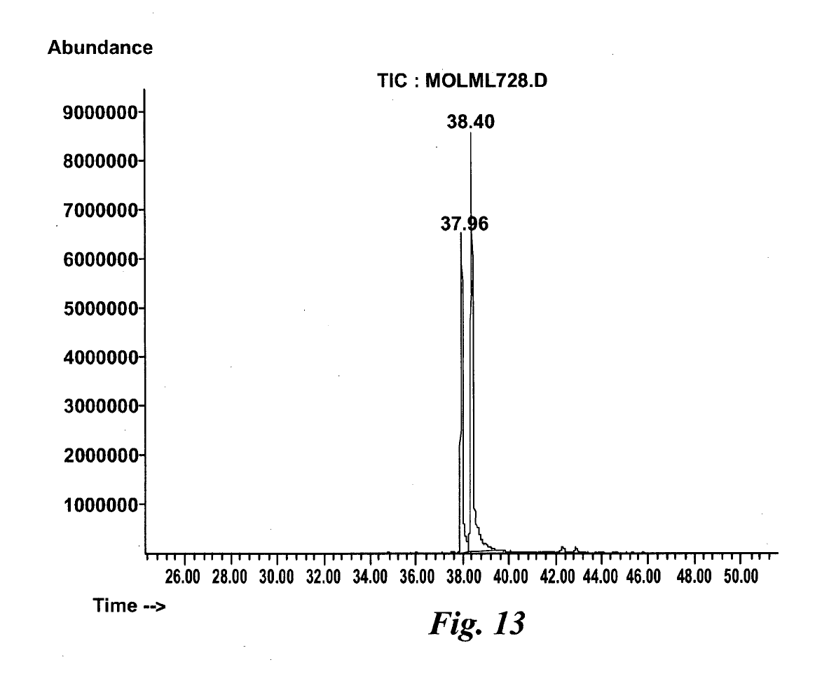

FIG. 13—Purity of chemical analogues by gc-ms: Javanol (sample B)

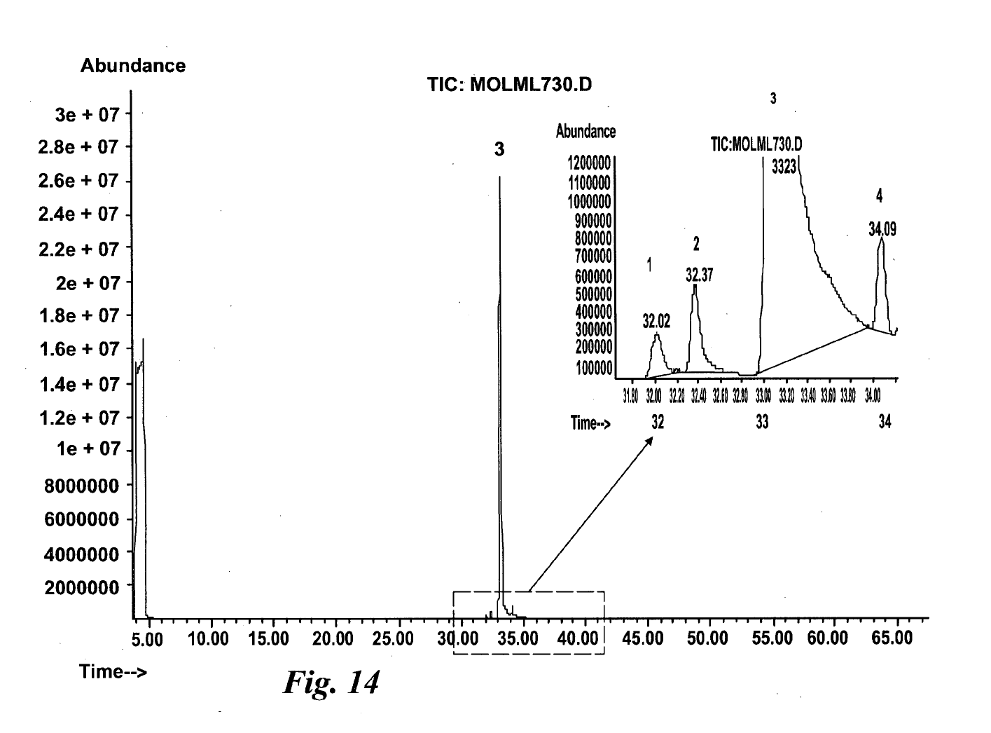

FIG. 14—Determination of Purity of Santaliff BHT by gc-ms (sample C)

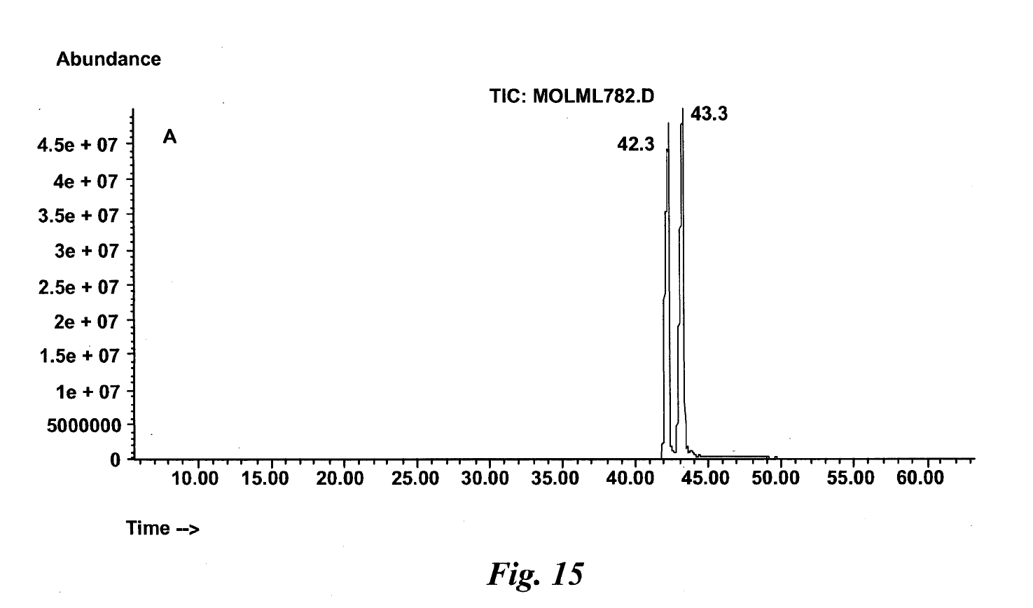

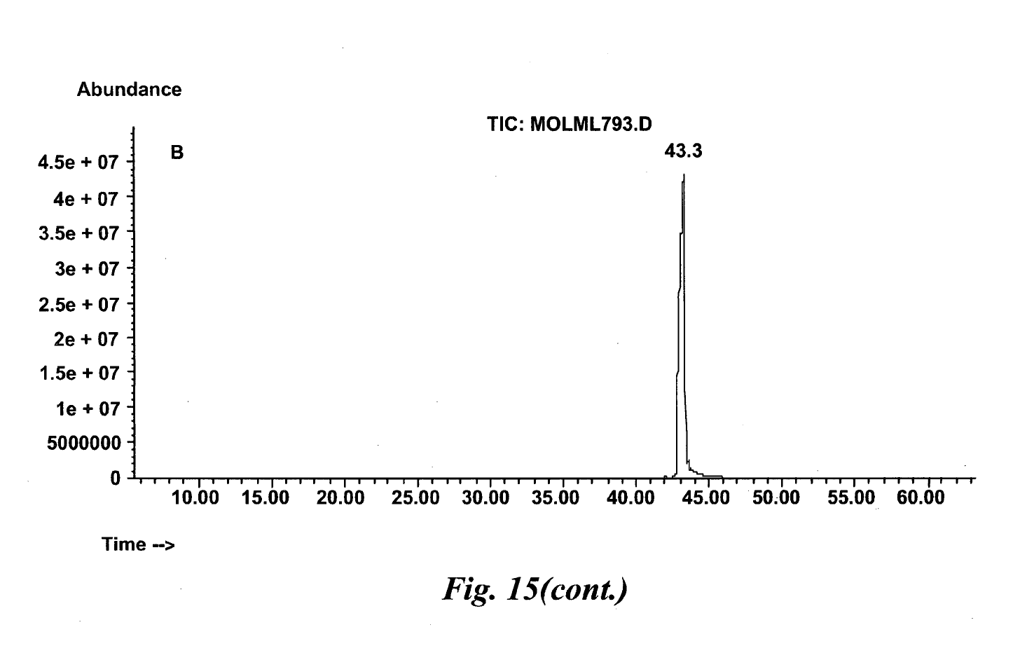

FIG. 15—Purity of farnesol (trans,trans) standards by gc-ms

### EXAMPLE 1

### Effects of Sandalwood Oil on Methane Production from Extracted Rumen Fluid

An initial screening of Sandalwood oil for it effects on volatile fatty acid and methane production in the rumen was carried out.

Thirty ml of a 33% buffered solution of rumen fluid withdrawn from a rumen canulated cow was incubated with 0.3 g of a 50:50 hay barley mix at 39° C. under anaerobic conditions.

After 16 hours the volume of gas produced and the percentage of methane in the headspace was measured and the resultant fermentation fluid was analysed for volatile fatty acids by HPLC.

Sandalwood oil, and control oils of commercial essential oil mixes or pure oils of eugenol or cinnamaldehyde were added in the amount of 500 μg/ml to the 30 ml buffered solution of rumen fluid prior to incubation. Sandalwood oil was obtained from Cardiff University and Sigma [SAFC (e.g. W30,050-0 lot no. 03722CC-396) and Sandalwood oil manufactured by Fluka (355263/1 lot no. 52706264).]

Table 1 shows the results of the methane production and HPLC analysis, and demonstrates that the Sandalwood oil when compared to the control experiments significantly decreased methane production and stimulated propionate production at the expense of acetate formation.

TABLE 1
Effect of essential oils (500 μg/ml) on total volatile
fatty acid and methane production by rumen fluid

| | | | | Total | |
|---|---|---|---|---|---|
| | | | | volatile | |
| | Acetate | Propionate | Butyrate | fatty acids | Methane |
| | formation μmole/g of feed | | | | |
| Cinnamaldehyde | 2832 | 492 | 73 | 3398 | 1471 |
| Eugenol | 2817 | 590 | 73 | 3479 | 1436 |
| Citral | 2754 | 534 | 72 | 3361 | 1461 |
| Essential oil mix 1 | 2816 | 479 | 72 | 3367 | 1455 |
| Essential oil mix 2 | 2559 | 460 | 77 | 3095 | 1411 |
| Essential oil mix 3 | 2727 | 485 | 97 | 3309 | 1480 |
| Sandalwood oil | 2266^{a} | 1083^{a} | 61 | 3410 | 1089^{a} |
| Control | 2768 | 519 | 75 | 3362 | 1446 |
| Standard error | 228.7*** | 99.1*** | 12.9*** | 211.3*** | 97.7*** |
| difference (SED) | | | | | |

^{a}= Different substrates significantly differing from the mean
***= P > 0.001

### EXAMPLE 2

### Influence of Sandalwood Oil on Breakdown of Labelled Bacteria

In a further screen the ability of the Sandalwood oil to prevent the breakdown of bacteria by protozoa in the rumen was tested using the methods described by Wallace and McPherson (1987) Br J Nutr., 58, pp 313-23. Briefly, the rumen bacterium Streptococcus bovis (S. bovis) was radio-labelled by growing it in a minimal media with ¹⁴C-lysine as the only available nitrogen source.

The labelled S. bovis were washed and then incubated anaerobically in rumen fluid at 39° C. for 3 h in the presence or absence of 500 μg/ml Sandalwood oil. The release of C¹⁴ was monitored by liquid scintillation spectrometry.

Sandalwood oil caused a significant decrease in the breakdown of the labelled S. bovis suggesting that addition of Sandalwood oil reduced bacterial protein turnover in the rumen (FIG. 1).

### EXAMPLE 3

### Rusitec Simulation

Further investigations of the action of Sandalwood oil were undertaken in the rumen simulation technique Rusitec. Rusitec was developed by Czerkawski and colleagues (Czerkawski and Breckenridge (1977) Br J Nutr. 38 , 371-84) as a long term simulation of rumen fermentation and has been used extensively to test feed additive for ruminants.

Simulation 1

The rumen-simulation technique (Rusitec) was used as described by Czerkawski and Breckenridge (1977).

The nominal volume in each reaction vessel was 850 ml and the dilution rate was set at 0.88 per day, the infused liquid being artificial saliva (McDougall, Biochem J 1948 43, 99-109) at pH 8.4. Inocula for the fermentation vessels were obtained from a pooled sample (liquid and particulate rumen contents) from three rumen-cannulated cattle fed on a conserved diet.

On the first day of the experiment 300 ml of strained rumen fluid, 300 ml of water and 300 ml of artificial saliva were placed in each reaction vessel. Solid rumen contents (80 g) were weighed into a nylon bag and one of these was placed inside the food container in each vessel together with a bag (20 g/d) of a basal diet of grass hay, barley, molasses, soyabean meal and a vitamin and mineral mixture. The food was provided in nylon bags, pore size 50 μm, which were gently agitated in the liquid phase. Two bags were present at any time and one bag was replaced each day to give a 48 h incubation.

The bags that were removed from the vessels were placed in plastic bags, and their contents washed and squeezed with 40 ml of artificial saliva. This was done twice for each bag, and the combined washings were poured back into the reaction vessels. Fermentation vessels were flushed with anaerobic grade CO₂ before filling, after filling, and then every day during feeding (when the nylon bags with the food were changed).

The duration of the experiment was 11 days, during which four vessels received Sandalwood oil (Fluka) (added the basal diet to reach an initial concentration of 333 μg/ml) the remaining vessels were controls.

Volatile fatty acids, ammonia and bacterial numbers were measured on days 10 and 11 of the experiment. On day 11 a non verotoxin containing strain of E. coli 0157 was added and it numbers traced over the last 24 hr of the experiment.

Simulation 2

In a second experiment a very similar protocol to simulation 1 was followed however 12 vessels were used and Sandalwood oil was added the basal diet to reach an initial concentrations of 0, 5, 50 or 500 μg/ml in triplicate vessels and the Sandalwood oil was sourced from Sigma Chemicals Ltd. The cattle used to provide the initial inoculum were grazing and a different source of hay and soyabean meal were used in the basal diet. The decline in the pathogen Listeria monocytogenes rather than E. coli 0157 was monitored.

TABLE 2
Effect of Sandalwood oil on daily volatile fatty acid
production in the rumen simulation fermentor Rusitec

| | Sandalwood oil concentration μg/ml | | |
|---|---|---|---|
| | 0 | 333 | SED |
| Acetate mmol/d | 16.2 | 15.1 | 0.90 |
| Propionate mmol/d | 12.2 | 12.1 | 0.92 |
| Butyrate mmol/d | 4.2 | 3.8 | 0.41 |
| Total VFA mmol/d | 32.6 | 30.9 | 2.10 |

TABLE 3
Effect of Sandalwood oil on daily ammonia and methane production
and bacterial numbers in the rumen simulation fermentor Rusitec

| | Sandalwood concentration μg/ml | | |
|---|---|---|---|
| | 0 | 333 | SED |
| Vessel pH | 6.8 | 6.8 | 0.03 |
| NH₃ mmol/d | 32.3 | 20.7 | 1.71 |
| CH₄ mmol/d | 1.0 | 0.77 | 0.131 |
| 24 h DM degradation % | 21.5 | 22.4 | 6.70 |
| 48 h DM degradation % | 35.1 | 35.4 | 6.03 |
| Bacteria counts | | | |
| Total/ml | 5.0 × 10⁹ | 6.0 × 10⁹ | 0.26 × 10⁹ |
| Log cellulolytic | 8.39 | 7.33 | 0.350 |
| (Back transformed | (2.45 × 10⁸) | (0.21 × 10⁸) | |
| means/ml) | | | |

TABLE 4
Effect of Sandalwood oil on daily volatile fatty acid
production in the rumen simulation fermentor Rusitec

| | Sandalwood Oil concentration (ug/ml) | | | | |
|---|---|---|---|---|---|
| | 0 | 5 | 50 | 500 | SED |
| Acetate mmol/d | 26.5 | 23.1 | 23.9 | 25.9 | 3.60 |
| Propionate mmol/d | 23.5 | 22.6 | 23.0 | 30.2 | 3.269 |
| Butyrate mmol/d | 11.1 | 7.9 | 10.2 | 8.8 | 3.09 |
| Total VFA mmol/d | 65.3 | 60.0 | 60.5 | 78.0 | 13.32 |

TABLE 5
Effect of Sandalwood oil on daily ammonia and methane production
and bacterial numbers in the rumen simulation fermentor Rusitec

| | Sandalwood Oil concentration (ug/ml) | | | | |
|---|---|---|---|---|---|
| | 0 | 5 | 50 | 500 | SED |
| Vessel pH | 6.7 | 6.7 | 6.7 | 6.7 | 0.05 |
| NH₃ mmol/d | 439 | 511 | 458 | 138 | 118.3 |
| CH₄ mmol/d | 4.5 | 2.7 | 2.4 | 2.1 | 0.35 |
| 24 h | 16.5 | 23.5 | 15.4 | 18.5 | 9.29 |
| degradation | | | | | |
| % | | | | | |
| 48 h | 38.6 | 32.7 | 26.7 | 30.0 | 5.44 |
| degradation | | | | | |
| % | | | | | |
| Bacteria | | | | | |
| counts | | | | | |
| Total/ml | 1 × 10⁹ | 1 × 10⁹ | 2 × 10⁹ | 3 × 10⁹ | 9.4 × 10⁸ |
| Cellulolytic/ | 8.6 × 10⁶ | 2.3 × 10⁶ | 8.7 × 10⁶ | 4.0 × 10⁶ | 3.49 × 10⁶ |
| ml | | | | | |

Sandalwood oil had no effect on VFA production in either experiment (Tables 2 and 4). Ammonia production was reduced by Sandalwood oil added at either 333 or 500 μg/ml but not lower concentrations (Tables 3 and 5).

Methane production was decreased by all concentration above 5 μg/ml (Tables 3 and 5). In the 1^{st} experiment 333 μg/ml of Sandalwood oil significantly reduced the survival of E. coli 0157 in the fermentor (FIG. 2) whilst in the second experiment Sandalwood oil at 50 or 500 μg/ml significantly reduced Listeria monocytogenes survival at 24 h after pathogen addition (FIG. 3).

Therefore, at between 50 and 500 μg/ml Sandalwood oil significantly reduced the production of methane an important greenhouse gas and also major energy loss from the animal. At concentrations above 333 μg/ml Sandalwood oil significantly decreased ammonia production suggesting a protein sparing effect. Sandalwood oil also significantly reduced the ability of the pathogens E. coli 0157 and Listeria monocytogenes to survive in the fermentor.

### EXAMPLE 4

### Methane Reduction in Response to Sandalwood Oil and Synthetic Sandalwood Oil Replacements

Thirty ml of a 33% buffered solution of rumen fluid withdrawn from a rumen canulated cow was incubated with 0.3 g of a 50:50 hay barley mix at 39° C. under anaerobic conditions

After 16 hours the volume of gas produced and the percentage of methane in the headspace was measured and the resultant fermentation fluid was analysed for volatile fatty acids by HPLC.

Sandalwood oil from either Fluka or SAFC and two different batches of Javanol (Givaudan) or a single batch of Santaliff (International Flavors & Fragrances) (both Javanol and Santaliff are artifical Sandalwood replacements) were added in the amount of 5, 50 or 100 or 500 pg/ml to the 30 ml buffered solution of rumen fluid prior to incubation.

FIG. 4 shows the results of the methane production, and demonstrates that the Sandalwood oil from Fluka and both batches of Javanol and Santalifff when compared to the control experiments significantly decreased methane.

### EXAMPLE 5

### Rusitec Experiments Using Sandalwood Oil and Synthetic Sandalwood Oil Replacements

The rumen-simulation technique (Rusitec) was used as described by Czerkawski and Breckenridge (1977).

The nominal volume in each reaction vessel was 850 ml and the dilution rate was set at 0.88 per day, the infused liquid being artificial saliva (McDougall, Biochem J 1948 43, 99-109) at pH 8.4. Inocula for the fermentation vessels were obtained from a pooled sample (liquid and particulate rumen contents) from three rumen-cannulated cattle fed on a conserved diet.

On the first day of the experiment 300 ml of strained rumen fluid, 300 ml of water and 300 ml of artificial saliva were placed in each reaction vessel. Solid rumen contents (80 g) were weighed into a nylon bag and one of these was placed inside the food container in each vessel together with a bag (20 g/d) of a basal diet of grass hay, barley, molasses, soyabean meal and a vitamin and mineral mixture. The food was provided in nylon bags, pore size 50 μm, which were gently agitated in the liquid phase. Two bags were present at any time and one bag was replaced each day to give a 48 h incubation.

The bags that were removed from the vessels were placed in plastic bags, and their contents washed and squeezed with 40 ml of artificial saliva. This was done twice for each bag, and the combined washings were poured back into the reaction vessels. Fermentation vessels were flushed with anaerobic grade CO₂ before filling, after filling, and then every day during feeding (when the nylon bags with the food were changed).

The duration of the experiment was 19 days, during which four vessels received Sandalwood oil (added the basal diet to reach an initial concentration of 100 μg/ml) four vessels received Javanol (added the basal diet to reach an initial concentration of 100 μg/ml) the remaining vessels were controls.

Volatile fatty acids, methane and bacterial numbers were measured on days 18 and 19 of the experiment. On day 19, Listeria inocula was added and it numbers traced over the last 24 hr of the experiment.

TABLE 6

| | Control | Javanol | Sandalwood | SED |
|---|---|---|---|---|
| pH | 6.70 | 6.66 | 6.67 | 0.022 |
| CH₄ mmol/d | 3.42 | 2.51 | 2.36 | 0.365 |
| 24 h DM degradation (5 | 21.7 | 24.7 | 23.9 | 3.47 |
| 48 g DM degradation (%) | 28.4 | 33.6 | 31.8 | 5.10 |
| Bacteria counts | | | | |
| Total × 10⁹/ml | 6.1 | 4.9 | 7.6 | 2.69 |
| Cellulolytic × 10⁶/ml | 1.6 | 1.9 | 1.8 | 0.25 |

TABLE 7

| | Control | Javanol | Sandalwood | SED |
|---|---|---|---|---|
| Acetate mmol/d | 21.2 | 23.9 | 21.4 | 2.70 |
| Propionate mmol/d | 14.5 | 16.7 | 13.4 | 1.86 |
| Butyrate mmol/d | 5.8 | 4.4 | 4.6 | 1.89 |
| Total VFA mmol/d | 41.5 | 45.0 | 39.4 | 2.83 |

Sandalwood oil and Javanol had no effect on VFA production. Methane production was decreased by both Sandalwood oil and Javanol. Sandalwood oil but not Javanol tended (P>0.08) to decrease Listeria monocytogenes survival at 24h after pathogen addition (FIG. 5).

### EXAMPLE 6

### Chemical Analysis of Sandalwood Oils and Synthetic Sandalwood Oil Replacements Used in Examples

Materials and Methods

Plant Extracts and Chemical Analogues

The sandalwood oil used was sourced from Swiss Herbal Remedies (B/N 540), Sigma-Aldrich Fine Chemicals Limited (SAFC) and Fluka (Dorset).

Chemical analogues based on the chemical structure of β-santalol, include Santaliff™ (supplier International Flavour and Fragrances, lot no: 8000485362) and Javanol™ (supplier Givaudan, lot no's: 9000591570 and 90000635339)—see FIGS. 7 and 8 for their chemical structures.

Farnesol (trans,trans) was purchased from Sigma-Aldrich (Poole, Dorset).

Sample Preparation

Sandalwood Oils

A 3 μl aliquot of sandalwood oil was transferred to a glass container and 1 ml of absolute ethanol added. The mixture was vortex mixed for one minute. A 100 μl aliquot of this mixture was transferred to another glass container and 300 μl of ethanol added, yielding a final sandalwood concentration of 0.075% v/v

Chemical Analogues

A 5 ul aliquot of sample was transferred to a glass container and 5 ml of absolute ethanol added giving a final concentration of 0.1% v/v.

Farnesol Standards

3 μl of farnesol was transferred to a glass container containing 1 ml of absolute ethanol added and vortex mixed for one minute yielding a final concentration of 0.3% v/v.

Gas Chromatography-Mass Spectrometry (GC-MS)

Samples were analysed using an Agilent Technologies 6890N gas chromatograph equipped with an Agilent 5973 Network mass selective detector and an Agilent 7683 series autosampler. A non-polar Phenomenex ZB5MS fused silica capillary column (supplier: Phenomenex) was used with the following dimensions: 30 m×0.25 mm id. and 0.25 μm film thickness.

The oven temperature was programmed from 50° C. to 240° C. at a rate of 3° C. min⁻¹ and maintained at this final temperature for five minutes. The helium carrier gas was set at a flow-rate of 0.6 ml min⁻¹ and maintained under constant pressure. The injector, source and mass transfer lines were set at temperatures of 250° C., 230° C. and 280° C. respectively.

The mass detector was used in the positive electron impact ionisation mode (El+) using an ionisation voltage of 70 eV. A scan range of 35 to 450 mass units was used for acquiring the mass spectra data with a sampling time of 2 which corresponds to 3.5 scans per second. Data acquisition was performed using the MSD Chemstation™ computer software. The injection port was configured for on-column Injections, hence, low sample volumes (0.2 μL) were used for all test samples and injected in the splitless mode. An ethanol solvent wash was included between sample injections and a solvent delay of three minutes applied to the mass detector.

The identification of the individual peaks were made by:

i) Comparing sample mass spectra to those stored in the NIST library database and

ii) Comparing sample mass spectra to published literature values.

The NIST libraries contain over 54,000 spectra. A reverse fit method was used for identification throughout. This method normalises data to 1000, hence compounds with library fits greater than 900 have a very high likelihood of being correctly assigned.

TABLE 8
Sandalwood oil (Swiss Herbal Remedies brand)

| Peak | RT | | Library Fit | | % Peak |
|---|---|---|---|---|---|
| No. | (Mins) | Compound | (Reverse) | Peak Area | Area |
| 1 | 30.47 | α-Santalene | 961 | 217084066 | 0.60 |
| | 31.07 | α-Bergamotene | 968 | 71342373 | 0.19 |
| 2 | 31.71 | epi-β-Santalene | 954 | 122464940 | 0.33 |
| | 32.24 | β-Santalene | 953 | 214940582 | 0.59 |
| | 33.20 | Curcumene | 980 | 216370397 | 0.59 |
| 3 | 36.92 | Dendrolasin | 920 | 585451408 | 1.62 |
| 4 | 41.51 | α-Santalol | 948 | 7152370863 | 19.79 |
| 5 | 41.96 | α-trans-Bergamotol | 935 | 3809882649 | 10.54 |
| 6 | 42.4 | epi-β-Santalol | 939 | 62990558 | 1.91 |
| 7 | 43.12 | trans-β-Santalol* | 902 | 7289614401 | 20.17 |
| 8 | 43.49 | Nuciferol | 933 | 4408086672 | 12.19 |
| 9 | 44.48 | Nuciferol isomer | 833 | 1658581138 | 4.58 |
| 10 | 44.71 | cis-Lanceol | 927 | 1083461495 | 2.99 |
| 11 | 50.68 | Unknown | — | 991883190 | 2.74 |
| | | | Total Area | 36138975840 | |
| | | | Compounds | | 74.47 |
| | | | Identified (%) | | |
| | | | Total alcohol | | 41.87 |
| | | | content (%) | | |

Total alcohol content expressed as santalol is 41.87%

TABLE 9
Sandalwood Oil (SAFC brand)

| Peak | RT | | Library Fit | | % Peak |
|---|---|---|---|---|---|
| \|No. | (Mins) | Compound | (Reverse) | Peak Area | Area |
| 1 | 30.46 | α-Santalene | 964 | 176,600,110 | 0.66 |
| | 31.06 | α-Bergamotene | 966 | 27,925,579 | 0.10 |
| 2 | 31.70 | epi-β-Santalene | 945 | 214,178,420 | 0.80 |
| 3 | 32.24 | β-Santalene | 963 | 323,842,042 | 1.21 |
| | 33.20 | Curcumene | 969 | 40,191,154 | 0.15 |
| 4 | 41.68 | α-Santalol | 947 | 13,388,942,510 | 50.12 |
| 5 | 41.99 | α-trans-Bergamotol | 952 | 1,726,157,246 | 6.46 |
| 6 | 42.53 | epi-β-Santalol | 950 | 1,385,388,247 | 5.186 |
| 7 | 43.21 | trans-β-Santalol | 953 | 7,344,111,142 | 27.49 |
| | 43.31 | Nuciferol | 941 | trace | — |
| 8 | 43.83 | Santalol isomer | 967 | 527,459,686 | 1.97 |
| 9 | 44.02 | Unknown | — | 373576932 | 1.39 |
| 10 | 44.58 | cis-Lanceol | 966 | 452,046,537 | 1.69 |
| | | | Total Area | 26,712,032,225 | |
| | | | Compounds | | 95.83 |
| | | | Identified (%) | | |
| | | | Total alcohol | | 84.77 |
| | | | content (%) | | |

Total alcohol content expressed as santalol is 84.77%

TABLE 10
Sandalwood Oil (Fluka brand)

| Peak | RT | | Library Fit | | % Peak |
|---|---|---|---|---|---|
| No. | (Mins) | Compound | (Reverse) | Peak Area | Area |
| 1 | 30.46 | α-Santalene | 967 | 158,399,943 | 1.07 |
| | 31.07 | α-Bergamotene | 968 | 27,597,223 | 0.18 |
| 2 | 31.70 | epi-beta-Santalene | 950 | 170,472,890 | 1.15 |
| 3 | 32.24 | β-Santalene | 959 | 258,738,060 | 1.75 |
| 4 | 33.11 | Unknown | — | 171,605,164 | 1.16 |
| | 33.22 | Curcumene | 894 | Trace | — |
| 5 | 41.53 | α-Santalol | 942 | 7,926,340,551 | 53.83 |
| 6 | 41.87 | α-trans-Bergamotol | 949 | 804,435,867 | 5.46 |
| 7 | 42.41 | epi-β-Santalol | 953 | 679,521,124 | 4.61 |
| 8 | 43.04 | trans-β-Santalol | 960 | 3,189,548,425 | 21.66 |
| 9 | 43.19 | Nuciferol | 949 | 197,251,727 | 1.34 |
| | 43.51 | Santalol isomer | 822 | 42,100,101 | 0.28 |
| 10 | 43.72 | Santalol isomer | 968 | 168,818,533 | 1.14 |
| 11 | 44.54 | cis-Lanceol | 961 | 129,590,895 | 0.88 |
| 12 | *50.91 | Diisoctyl phthalate | 951 | — | — |
| | | (plasticizer impurity) | | | |
| | | | Total Area | 14,725,166,618 | |
| | | | Compounds | | 93.35 |
| | | | Identified (%) | | |
| | | | Total alcohol | | 81.52 |
| | | | content (%) | | |

Total alcohol content expressed as santalol is 81.52%.

TABLE 11
Purity of chemical analogues by gc-ms: Javanol (sample A)

| Peak | RT | | Peak | % |
|---|---|---|---|---|
| No. | (Mins) | Compound | Area | Peak Area |
| 1 | 38.00 | Javanol Isomer 2 | 791441868 | 40.82 |
| 2 | 38.44 | Javanol Isomer 2 | 1124046450 | 57.98 |
| 3 | 42.268 | Unknown | 22946317 | 1.18 |
| | | Total Area | 1938434635 | |

Total % Purity = 98.62%

TABLE 12
Purity of chemical analogues by gc-ms: Javanol (sample B)

| Peak | RT | | Peak | % |
|---|---|---|---|---|
| No. | (Mins) | Compound | Area | Peak Area |
| 1 | 37.95 | Javanol Isomer 1 | 406015504 | 37.63 |
| 2 | 38.41 | Javanol Isomer 2 | 651570188 | 60.39 |
| 3 | 42.27 | Unknown 1 | 13370100 | 1.23 |
| 4 | 42.88 | Unknown 2 | 7962910 | 0.73 |
| | | Total Area | 1078918703 | |

Total % Purity = 98.02%

TABLE 13
Determination of Purity of Santaliff BHT by gc-ms (sample C)

| Peak | RT | | Library Fit | | % Peak |
|---|---|---|---|---|---|
| No. | (Mins) | Compound | (Reverse) | Peak Area | Area |
| 1 | 32.01 | Unknown | | 16811049 | 0.72% |
| 2 | 32.37 | Santaliff isomer | | 31247462 | 1.35% |
| 3 | 33.22 | Santaliff | — | 2238211688 | 96.80% |
| 4 | 34.08 | Butylated hydroxy | 894 | 25731644 | 1.11% |
| | | toluene* | | | |
| | | Total Area | | 2312001843 | |

Total % Purity = 98.15%

Summary of Results

The santalol isomers were found to be the major chemicals present in the sandalwood oil obtained from India and Indonesia, showing a total santalol content between 78.1-84.7%.

The sandalwood oil procured from the Asia-Pacific/Australia region contained much lower amounts of santalol (mean 41.9%) with substantially high levels of α-trans-Bergamotol (mean 10.4%) and nuciferol (mean 12.6%) compared to sandalwood oil samples obtained from India and Indonesia. Oil from these latter two countries contained a-trans-Bergamotol and nuciferol at lower levels (5.4-7.6%) and (1.3-1.6%) respectively.

The proportion of the two major santalol isomers (α and β), in the various sandalwood oils, varied greatly and favoured the α-isomer in sandalwood oil samples from India and Indonesia (approximately 2:1), whilst slightly favouring the β-isomer for the oils obtained from the Asia-Pacific /Australia region (mean 0.95: 1).

The sandalwood oil procured from the Asia-Pacific/Australia region (Swiss Herbal Remedies) was the only oil found to contain the furano-sesquiterpene dendrolasin.

The chemical analogues santaliff and javanol showed a mean purity of 98.15% and 98.32%, respectively, when analysed by gc-ms.

Analysis of the farnesol standards (old and new) showed the presence of the cis,trans farnesol isomer to substantially increase with time. Both farnesol standards showed greater than 96% purity for the sum of isomers.

Discussion of Results

All the sandalwood oils, tested, contained the same general bouquet of chemicals known as sesquiterpenes. These comprised of α-santalol, β-santalol, bergamotol, epi-santalol, nuciferol and lanceol. Their abundance in sandalwood oil was found to vary and depended upon the geographical location from which the oils were sourced.

GC-MS was able to classify the sandalwood oils into two chemical groups, according to their santalol content. Those oils containing high total santalol levels (Fluka and SAFC) and can be assured to be authentic sandalwood oil from the species S. album

The low santalol level and high nuciferol content detected for the oil from Swiss Herbal Remedies, however, suggests its origin to be either from S. spicatum, a species of sandalwood indigenous to Western Australia or S. austrocaledonicum from the Pacific Islands. S. spicatum has been reported to contain high a farnesol content (Jirovetz et al., 2006), which was not confirmed in these oils. It is possible, however, that the farnesol peak (retention time 42-43 minutes) was co-eluting with the β-santalol peak (42.8-43 minutes). Repeat analysis on a more polar chromatography column would probably separate these compounds and confirm its presence. The detection of dendrolasin in the two samples of sandalwood oil labelled Swiss Herbal Remedies (Asia Pacific/Australia region) confirmed their origin to be from the sandalwood species S. spicatum indigenous to Australia.

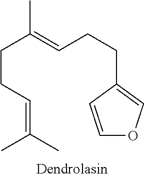

### EXAMPLE 7

### Effect of Further Sandalwood Analogues

Further testing of sandalwood analogues was conducted following the protocols described in Examples 3 and 5.

The sandalwood analogues (chemical structures are shown in FIGS. 7 and 8) tested were:

Sanjinol

Bacdanol

Santaliff

Sandela

Javanol

Ebanol

Sandalore

Javanol (batch no. 9000699712), Sandalore (batch no. 9000703989), Ebanol (batch no. 900068333) and Sandela (batch no. 9000701064) were obtained from either Givaudan (UK) (via S. Black Limited, Hertford, UK) and Santaliff, Bacdanol and Sanjinol were obtained from International Flavour and Fragrances (Haverhill, UK)

Results

TABLE 14

| | μg/ml | Acetate | Propionate | Butyrate | TVFA | Methane |
|---|---|---|---|---|---|---|
| Compound | μmol/g of feed added | | | | | |
| Sanjinol | 5 | 1978 | 426 | 448 | 2852 | 1107 |
| Sanjinol | 50 | 2192 | 579 | 466 | 3237 | 835 |
| Sanjinol | 100 | 2359 | 697 | 512 | 3568 | 795 |
| Sanjinol | 500 | 1849 | 744 | 225 | 2818 | 741 |
| Bacdanol | 5 | 2037 | 444 | 438 | 2919 | 934 |
| Bacdanol | 50 | 2049 | 434 | 458 | 2942 | 1047 |
| Bacdanol | 100 | 2419 | 515 | 514 | 3448 | 995 |
| Bacdanol | 500 | 2331 | 864 | 309 | 3503 | 942 |
| Santaliff | 5 | 2174 | 466 | 458 | 3098 | 1031 |
| Santaliff | 50 | 2142 | 442 | 490 | 3074 | 1052 |
| Santaliff | 100 | 2301 | 543 | 462 | 3305 | 844 |
| Santaliff | 500 | 2101 | 741 | 296 | 3138 | 801 |
| Sandela batch no. 9000701064 | 5 | 2223 | 465 | 482 | 3169 | 996 |
| Sandela batch no. 9000701064 | 50 | 2235 | 450 | 513 | 3198 | 1052 |
| Sandela batch no. 9000701064 | 100 | 2297 | 477 | 515 | 3289 | 1078 |
| Sandela batch no. 9000701064 | 500 | 2270 | 680 | 317 | 3267 | 928 |
| Javanol batch no. 9000699712 | 5 | 2401 | 493 | 538 | 3432 | 978 |
| Javanol batch no. 9000699712 | 50 | 2622 | 524 | 593 | 3739 | 904 |
| Javanol batch no. 9000699712 | 100 | 2504 | 542 | 499 | 3545 | 859 |
| Javanol batch no. 9000699712 | 500 | 2045 | 833 | 229 | 3107 | 744 |
| Ebanol batch no. 9000683337 | 5 | 2136 | 427 | 488 | 3051 | 952 |
| Ebanol batch no. 9000683337 | 50 | 2129 | 436 | 474 | 3039 | 934 |
| Ebanol batch no. 9000683337 | 100 | 2430 | 533 | 458 | 3421 | 833 |
| Ebanol batch no. 9000683337 | 500 | 2333 | 939 | 332 | 3604 | 683 |
| Sandalore batch no. 9000703989 | 5 | 2118 | 431 | 487 | 3036 | 951 |
| Sandalore batch no. 9000703989 | 50 | 1972 | 409 | 453 | 2834 | 1086 |
| Sandalore batch no. 9000703989 | 100 | 2119 | 471 | 432 | 3023 | 1174 |
| Sandalore batch no. 9000703989 | 500 | 2249 | 897 | 305 | 3451 | 902 |
| Control | | 1940 | 526 | 477 | 2943 | 1054 |
| SED | | 198.7 | 49.33** | 40.93 | 270.1 | 83.9*** |

TABLE 15

| BOTTLE | | | compound rate | CH4 | |
|---|---|---|---|---|---|
| NO. | rep | Compound no. | ug/30 ml | umol/g | |
| 1 | a | 200 ul ETOH | 0 | 1153.037 | |
| 2 | b | 200 ul ETOH | 0 | 1341.099 | |
| 3 | c | 200 ul ETOH | 0 | 1034.304 | 1176.147 |
| 4 | a | 200 ul DMSO | 0 | 968.2334 | |
| 5 | b | 200 ul DMSO | 0 | 951.8643 | |
| 6 | c | 200 ul DMSO | 0 | 927.0059 | 949.0346 |
| 7 | a | Javanol batch no. 9000699712 | 150 | 956.9591 | |
| | | in 200 ul DMSO | | | |
| 8 | b | Javanol batch no. 9000699712 | 150 | 888.4071 | |
| | | in 200 ul DMSO | | | |
| 9 | c | Javanol batch no. 9000699712 | 150 | 895.6242 | 913.6635 |
| | | in 200 ul DMSO | | | |
| 10 | a | Javanol batch no. 9000699712 | 15000 | 647.283 | |
| | | in 200 ul DMSO | | | |
| 11 | b | Javanol batch no. 9000699712 | 15000 | 563.0216 | |
| | | in 200 ul DMSO | | | |
| 12 | c | Javanol batch no. 9000699712 | 15000 | 605.5467 | 605.2838 |
| | | in 200 ul DMSO | | | |
| 13 | a | Javanol batch no. 9000699712 | 150 | 1403.051 | |
| | | in 200 ul ETOH | | | |
| 14 | b | Javanol batch no. 9000699712 | 150 | 1381.609 | |
| | | in 200 ul ETOH | | | |
| 15 | c | Javanol batch no. 9000699712 | 150 | 1335.905 | 1373.522 |
| | | in 200 ul ETOH | | | |
| 16 | a | Javanol batch no. 9000699712 | 15000 | 1145.346 | |
| | | in 200 ul ETOH | | | |
| 17 | b | Javanol batch no. 9000699712 | 15000 | 1230.103 | |
| | | in 200 ul ETOH | | | |
| 18 | c | Javanol batch no. 9000699712 | 15000 | 1099.314 | 1158.255 |
| | | in 200 ul ETOH | | | |
| 19 | a | Sanjinol in 200 ul DMSO | 150 | 1107.671 | |
| 20 | b | Sanjinol in 200 ul DMSO | 150 | 793.8945 | |
| 21 | c | Sanjinol in 200 ul DMSO | 150 | 914.1195 | 938.5618 |
| 22 | a | Sanjinol in 200 ul DMSO | 15000 | 615.569 | |
| 23 | b | Sanjinol in 200 ul DMSO | 15000 | 893.1559 | |
| 24 | c | Sanjinol in 200 ul DMSO | 15000 | 722.2742 | 743.6664 |
| 25 | a | Sanjinolin 200 ul ETOH | 150 | 1064.879 | |
| 26 | b | Sanjinolin 200 ul ETOH | 150 | 1232.288 | |
| 27 | c | Sanjinolin 200 ul ETOH | 150 | 1162.641 | 1153.269 |
| 28 | a | Sanjinolin 200 ul ETOH | 15000 | 1063.899 | |
| 29 | b | Sanjinolin 200 ul ETOH | 15000 | 1351.649 | |
| 30 | c | Sanjinolin 200 ul ETOH | 15000 | 1059.708 | 1158.419 |

The sandalwood analogues tested (tables 14 and 15) showed a generalised improvement in a variety of properties when administered to the rusitec model. In particular, reductions in methane were achieved and these were shown to be dose specific reductions, such that increased doses of the sandalwood analogue are generally associated with a decrease in methane and volatile fatty acid production.

1. The use of a sandalwood extract or a sandalwood analogue as an additive to animal foodstuff.

2. The use of claim 1 wherein the extract or analogue is added to the foodstuff after the foodstuff has been prepared.

3. The use of claim 1 wherein the extract or analogue is added to the foodstuff during preparation of the foodstuff.

4. The use of claim 1 wherein the amount of sandalwood extract or analogue used is 0.025 and 50 g per day.

5. The use of claim 4 wherein the amount is between 0.5 and 50 g per day for large ruminants and horses and 0.025 g-2.5 g per day in smaller ruminants.

6. The use of claim 5 wherein the amount is 5 g per day for large ruminants and horses.

7. The use of claim 5 wherein the amount is 2.5 g per day for small ruminants

8. The use of claim 1 wherein the amount of sandalwood extract or analogue used is between 25 mg/kg-50 g/kg.

9. The use of claim 8 wherein the amount is 500 mg/kg.

10. An animal foodstuff comprising a sandalwood extract or a sandalwood analogue.

11. The foodstuff of claim 10 comprising between 0.025 and 50 g per day of sandalwood extract or analogue.

12. The foodstuff of claim 11 wherein the amount is between 0.5 and 50 g per day for large ruminants and horses and 0.025 g-2.5 g per day in smaller ruminants.

13. The foodstuff of claim 12 wherein the amount is 5 g per day for large ruminants and horses.

14. The foodstuff of claim 12 wherein the amount is 2.5 g per day for small ruminants

15. The foodstuff of of claim 10 wherein the amount of sandalwood extract used is between 25 mg/kg-50 g/kg.

16. The foodstuff of claim 10 wherein the amount is 500 mg/kg.

17. The foodstuff of claim 10 packaged and presented for feeding a ruminant or a horse.

18. A method for reducing the growth of pathogenic bacteria in the digestive system of a ruminant or horse comprising supplying the ruminant or horse with a sandalwood extract or a sandalwood analogue.

19. The method of claim 18 wherein pathogenic bacterial growth is reduced in the rumen.

20. The method of claim 18 wherein the bacteria is E. coli and/or Listeria monocytogenes.

21. The method of claim 18 wherein the ruminant or horse is supplied with an animal foodstuff comprising a sandalwood extract or a sandalwood analogue.

22. A method of increasing meat and/or milk production from a ruminant or horse comprising supplying the ruminant or horse with a sandalwood extract or a sandalwood analogue.)

23. The method of claim 22 wherein the ruminant or horse is supplied with an animal foodstuff comprising a sandalwood extract or a sandalwood analogue.

24. A method for reducing protein breakdown in the digestive system of a ruminant or horse comprising supplying the ruminant or horse with sandalwood extract.

25. The method of claim 24 wherein protein breakdown is reduced in the rumen.

26. The method of claim 24 wherein the ruminant or horse is supplied with an animal foodstuff comprising a sandalwood extract or a sandalwood analogue.

27. A method of reducing methane emission by a ruminant or horse comprising supplying the ruminant or horse with sandalwood extract.

28. The method of claim 27 wherein the ruminant or horse is supplied with an animal foodstuff comprising a sandalwood extract or a sandalwood analogue.

29-32. (canceled)

33. The use of claim 1 in which the sandalwood analogue has the structure: 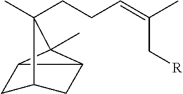
where:
R═OH.

34. The use of claim 1 in which the sandalwood analogue has the structure: 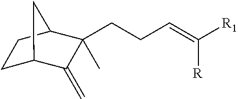
where
R═CH₂OH and R1═H; or
R═H and R1═CH₂OH.

35. The use of claim 1 in which the sandalwood analogue has the structure: 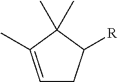
where:
R=3 methyl pentanol, 3-methyl pent-4-en-2-ol, (E)-2-methylbut-2-en-1-ol, or (E)-2-ethylbut-2-en-1-ol.

36-38. (canceled)

39. The foodstuff of claim 10 in which the sandalwood analogue has the structure: 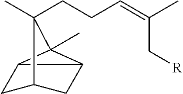
where:
R═OH.

40. The method of claim 18 in which the sandalwood analogue has the structure: 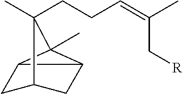
where:
R═OH.

41. The foodstuff of claim 10 in which the sandalwood analogue has the structure: 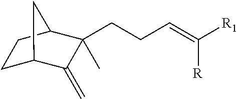
where
R═CH₂OH and R1═H; or
R═H and R1═CH₂OH.

42. The method of claim 18 in which the sandalwood analogue has the structure: 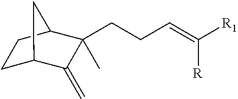
where
R═CH₂OH and R1═H; or
R═H and R1═CH₂OH.

43. The foodstuff of claim 10 in which the sandalwood analogue has the structure: 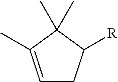
where:
R=3 methyl pentanol, 3-methyl pent-4-en-2-ol, (E)-2-methylbut-2-en-1-ol, or (E)-2-ethylbut-2-en-1-ol.

44. The method of claim 18 in which the sandalwood analogue has the structure: 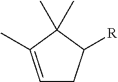
where:
R=3 methyl pentanol, 3-methyl pent-4-en-2-ol, (E)-2-methylbut-2-en-1-ol, or (E)-2-ethylbut-2-en-1-ol.
